(19) United States
(12) Patent Application Publication
(10) Pub. No.: US 2021/0241207 A1
(43) Pub. Date: Aug. 5, 2021

(54) METHOD FOR NOTIFYING OF EXPIRY DATE OF MEDICINE AND COMPUTER PROGRAM RECORDING MEDIUM THEREFOR

(71) Applicant: E BLUE CO., LTD., Gumi-si (KR)

(72) Inventor: Nahyeon LEE, Ulsan (KR)

(21) Appl. No.: 16/761,328

(22) PCT Filed: Nov. 5, 2018
(86) PCT No.: PCT/KR2018/013327

§ 371 Date: Mar. 24, 2021

(30) Foreign Application Priority Data
Nov. 6, 2017 (KR) 10-2017-0146931

(51) Int. Cl. G06Q 10/08; G16H 20/10; G06Q 20/20

(52) U.S. Cl. CPC G06Q 10/087; G06Q 20/20; G16H 20/10

(57) ABSTRACT

The present invention relates to a method of providing notification of an expiration date of a medicinal product and a recording medium on which a computer program for performing the method is recorded. According to an aspect of the present invention, there is provided a method of providing notification of an expiration date of a medicinal product, which is performed in a pharmacy system that includes a POS terminal and an audio output unit, the method including 1) determining whether or not a medicinal product subject to notification, of which an expiration date satisfies a preset notification condition, is present, and 2) outputting information about the medicinal product subject to notification in the form of audio guidance through the audio output unit.

### TECHNICAL FIELD

The present invention relates to a method of providing notification of an expiration date of a medicinal product and a recording medium on which a computer program for performing the method is recorded. More particularly, the present invention relates to a method of providing notification of an expiration date of a medicinal product, which is capable of outputting information about a medicinal product of which an expiration date is approaching, in the form of audio guidance to a pharmacist and thus facilitating medicinal product inventory management in a pharmacy and minimizing the number of unsalable medicinal products in inventory by exchanging medicinal product inventory information with another pharmacy for assignment through an agent system, and to a recording medium on which a computer program for performing the method is recorded.

### BACKGROUND ART

Many types of medicinal products including prescription medicinal products and over-the-counter medicinal products are sold in pharmacies. However, it is a financial burden to an individual pharmacy to keep all these medicinal products in stock. Therefore, generally, the individual pharmacy keeps in stock medicinal products that are expected to be prescribed by doctors who work for a nearby hospital or a doctor's office or commonly used over-the-counter medicinal products.

Nevertheless, when a doctor who works for a nearby hospital or a doctor's office changes a prescription or when the number of patients that make a visit to a pharmacy decreases, the pharmacy cannot help but keep many unsold medicinal products in stock or dispose of them.

A pharmacy that keeps various types of medicinal products in stock needs to employ a system for efficient medicinal product inventory management in order to alleviate the pharmacist's burden of managing the inventory.

An example of the related art is Korean Patent No. 10-1204647 titled “APPARATUS AND METHOD FOR MANAGING INVENTORY OF MEDICINAL PRODUCTS USING MEDICINAL PRODUCT IDENTIFICATION INFORMATION”, which is directed to medicinal product inventory management.

In the related art, in order to easily manage information on an inventory of medicinal products, an apparatus for managing an inventory of medicinal products using medicinal product identification information is configured to include an interface and a control processor. The interface operates in conjunction with input of the medicinal product identification information from a medicinal product inventory server and thus searches a database for order information about the medicinal product inventory server. When the medicinal product identification information that is input is identified from the order information that is found as the result of the search, the control processor provides the order information to the medicinal product inventory server and thus updates the medicinal product inventory server with medicinal product inventory information.

However, in the related art, because only an inventory of medicinal products is managed, there is a problem in that expiration dates (shelf lives) of medicinal products are not managed at all.

Therefore, when a pharmacy cannot sell medicinal products because expiration dates thereof expires, this can be the financial burden to the pharmacy. In order to prevent this situation, a pharmacist can check on a daily basis whether or not expiration dates of many medicinal products expire. However, this also causes the problem of increasing rather than decreasing the pharmacist's workload.

### DISCLOSURE

### Technical Problem

An objective of the present invention, which was conceived to solve the problems described above, is to provide a method of providing notification of an expiration date of a medicinal product, which is capable of outputting information about a medicinal product of which an expiration date is approaching, in the form of audio guidance to a pharmacist and thus facilitating medicinal product inventory management in a pharmacy and minimizing the number of unsalable medicinal products in inventory by exchanging medicinal product inventory information with another pharmacy for assignment through an agent system, and to a recording medium on which a computer program for performing the method is recorded.

### Technical Solution

According to an aspect of the present invention, there is provided a method of providing notification of an expiration date of a medicinal product, which is performed in a pharmacy system that includes a POS terminal and an audio output unit, the method including: 1) determining whether or not, among medicinal products registered as inventory, there is a medicinal product subject to notification, of which an expiration date satisfies a preset notification condition; and 2) outputting information about the medicinal product subject to notification in the form of audio guidance through the audio output unit.

In the method, the step 1) may be automatically performed according to a preset guidance time condition.

In the method, the pharmacy system may further include an audio input unit, and the step 1) may be performed on the basis of audio that is input by a user through the audio input unit.

In the method, in the step 1), among the medicinal products registered as inventory, a medicinal product of which an expiration date falls within a preset notification period range may be determined as the medicinal product subject to notification that satisfies the notification condition.

In the method, in the step 1), at the time of a medicinal product sale to a specific patient, when, among the medicinal products registered as inventory, a medicinal product of which an expiration date is determined as exceeding a medication period for the specific patient occurs, the medicinal product may be determined as the medicinal product subject to notification that satisfies the notification condition.

The method, wherein the pharmacy system may further include a screen output unit, may further include 21) screen-displaying the information about the medicinal product subject to notification through the screen output unit.

In the method, the POS terminal may include a first human-body detection sensor configured to detect a pharmacist-side area and a second human-body detection sensor configured to detect a customer-side area, and the pharmacy system may perform the step 2) when the first human-body detection sensor detects a human body and the second human-body detection sensor does not detect the human body.

The method, wherein the pharmacy system may operate in conjunction with an agent system via a network, may further include 3) receiving input about a medicinal product subject to processing prior to an expiration date, among the medicinal products subject to notification about which the information is output in the form of audio guidance; 4) transmitting the information about the input medicinal product subject to processing prior to the expiration date to the agent system; 5) receiving information about another pharmacy system that makes a request for assignment of the medicinal product subject to processing prior to the expiration date, from the agent system; and 6) transmitting an approval of the assignment of the medicinal product subject to processing prior to the expiration date to the agent system, when the approval of the assignment of the medicinal product subject to processing prior to the expiration date is input for processing, the medicinal product subject to processing prior to the expiration date being requested for assignment.

In the method, in the step 4), at least one of a price condition for assigning the medicinal product and an expiration date condition for assigning the medicinal product may be included in the information about the medicinal product subject to processing prior to the expiration date.

According to another aspect of the present invention, there is provided a method of providing notification of an expiration date of a medicinal product, which is performed in a pharmacy system that includes a POS terminal and an audio output unit and operates in conjunction with an agent system via a network, the method including: 1) transmitting information about a medicinal product registered as inventory in the pharmacy system to the agent system, the agent system managing information on an inventory in each pharmacy system on the basis of the received information; and 2) when it is determined that, among medicinal products registered as inventory, there is a medicinal product subject to notification, of which the expiration date satisfies a preset notification condition, outputting information about the medicinal product subject to notification in the form of audio guidance through the audio output unit, the determination being made in the agent system and the information about the medicinal product subject to notification being transmitted from the agent system to the pharmacy system.

According to still another aspect of the present invention, there is provided a method of providing notification of an expiration date of a medicinal product, which is performed in an agent system that operates via a network in conjunction with a pharmacy system that includes a POS terminal and an audio output unit, the method including: 1) receiving information about a medicinal product registered as inventory from the pharmacy system and managing information on the inventory in each pharmacy system on the basis of the received information; 2) receiving information about a medicinal product subject to processing prior to an expiration date from the pharmacy system, the medicinal product subject to processing prior to the expiration date being a medicinal product input as a medicinal product subject to processing prior to the expiration date among medicinal products subject to notification, of which an expiration date satisfies a preset notification condition, and being medicinal products registered as inventory in each pharmacy system, and the medicinal product subject to processing prior to the expiration date being input on the basis of information about the medicinal product subject to notification, which is output in the form of audio guidance through the audio output unit in the pharmacy system; and 3) providing the information about the medicinal product subject to processing prior to the expiration date to another pharmacy system.

The method may further include 4) receiving information on a request for assignment of the medicinal product subject to processing prior to the expiration date from the another pharmacy system; and 5) providing the information on the request from the another pharmacy system to the pharmacy system that transmits the information about the medicinal product subject to processing prior to the expiration date.

In the method, in the step 3), the information about the medicinal product subject to processing prior to the expiration date is provided only to another pharmacy system that in advance registers its intention to receive the medicinal product.

In the method, in the step 3), the information about the medicinal product subject to processing prior to the expiration date may be provided only to another pharmacy system that, among other pharmacy systems that in advance register their intentions to receive the medicinal product, is located in the same area as the pharmacy system.

According to still another aspect of the present invention, there is provided a computer-readable recording medium on which a computer program for performing the method of providing notification of an expiration date of a medicinal product on a computer is recorded.

### Advantageous Effects

According to the present invention, with audio guidance, a pharmacist can know about a medicinal product of which an expiration date is approaching and manage the medicinal product. This provides the advantage that medicinal product inventory management is facilitated while decreasing the pharmacist's burden of managing a medicinal product inventory in association with expiration dates of medicinal products.

Particularly, according to the present invention, the information about the expiration date is output in the form of audio guidance. This provides the advantage that the audio guidance has a higher recognition rate than screen display guidance.

In addition, according to the present invention, medicinal product inventory information about a medicinal product, of which an expiration date is approaching, is exchanged with another pharmacy. This provides the advantage that facilitated and cost-saving medicinal product inventory management is possible while minimizing the number of unsalable medicinal products in inventory by mutually assigning and receiving necessary medicinal products.

### DESCRIPTION OF DRAWINGS

FIG. 1 is a diagram illustrating a configuration of a pharmacy system according to an embodiment of the present invention;

FIGS. 2 and 3 are flowcharts each illustrating a method of providing notification of an expiration date of a medicinal product according to an embodiment of the present invention;

FIG. 4 is a schematic diagram illustrating the inside of a pharmacy in which the pharmacy system according to the embodiment of the present invention is installed;

FIG. 5 is a diagram illustrating a configuration of a system in which a method of providing notification of an expiration date of a medicinal product according to another embodiment of the present invention is performed;

FIG. 6 is a diagram illustrating a configuration of an agent system according to an embodiment of the present invention;

FIG. 7 is a flowchart illustrating a method of providing notification of an expiration date of a medicinal product according to still another embodiment of the present invention;

FIG. 8 is a flowchart illustrating a method of providing notification of an expiration date of a medicinal product according to still another embodiment of the present invention;

FIG. 9 is a diagram illustrating a system in which a method of providing notification of an expiration date of a medicinal product according to still another embodiment of the present invention is performed;

FIG. 10 is a flowchart illustrating a method of providing notification of an expiration date of a medicinal product according to still another embodiment of the present invention; and

FIG. 11 is a diagram illustrating a pharmacy system in terms of hardware according to another embodiment of the present invention.

### BEST MODE MODE FOR INVENTION

The present invention can be implemented in various other forms without departing from its technical spirit or main characteristics. Therefore, the embodiments of the present invention are merely illustrative in all respects and should not be interpreted as limiting.

Terms such as first and second are used only for the purpose of distinguishing one component from other components. For example, the first component may be referred to as a second component without departing from the scope of the present invention, and similarly, the second component may be referred to as a first component.

When an element is said to be “connected” or “coupled” to other component, the element may be directly connected to or connected to the other component, but another components may exist in between.

The singular expression used in the present application includes the plural expression unless the context clearly indicates otherwise. In the present application, the terms “comprise”, “have”, or “include”, etc. are intended to express the existence of elements or combinations thereof described in the specification, and do not preclude the possibility that other elements or features are present or added.

Preferable embodiments of the present invention will be described in detail below with reference to the accompanying drawings.

When describing each of the embodiments of the present invention, a “system” refers to a system in a logical concept that serves as a server or client in a normal server-client environment, and for example, can be realized as a general computing apparatus (for example, a PC, a tablet PC, a smartphone, or a computer as a server) that is capable of using the web service or application service.

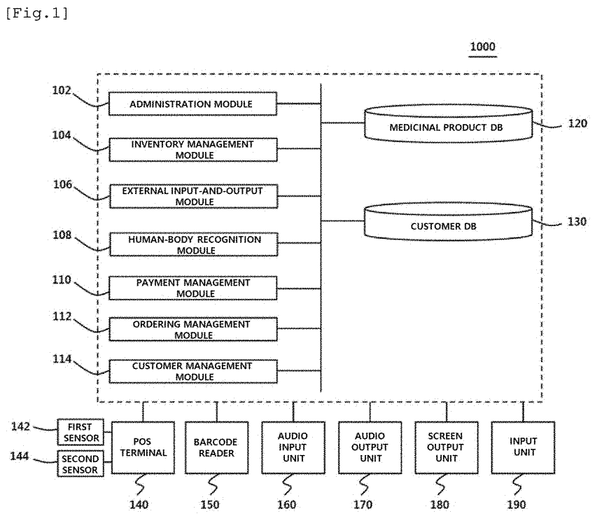

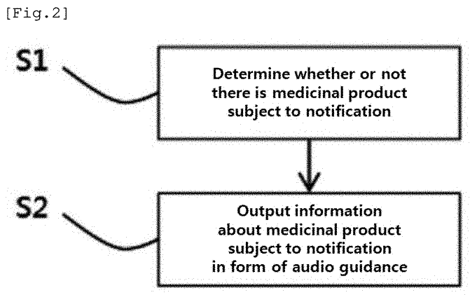

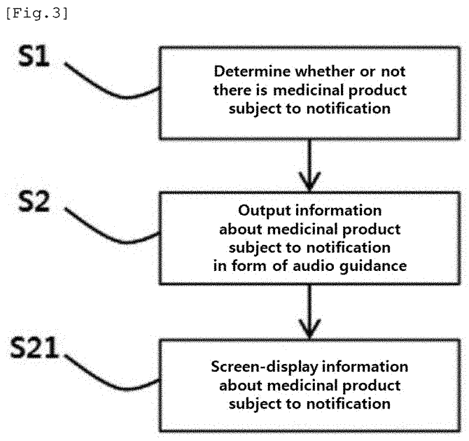

FIG. 1 is a diagram illustrating a configuration of a pharmacy system according to an embodiment of the present invention. FIGS. 2 and 3 are flowcharts each illustrating a method of providing notification of an expiration date of a medicinal product according to an embodiment of the present invention.

The method of providing notification of an expiration date of a medicinal product according to the embodiment of the present invention is performed in a pharmacy system 1000 that includes a POS terminal 140 and an audio output unit 170.

The pharmacy system 1000 includes the POS terminal 140 for accepting payment of a price of a medicinal product purchased by a customer, a barcode reader 150 for recognizing a medicinal product, an audio output unit 170 for outputting audio, and an input unit 190 for receiving input about a medicinal product subject to processing prior to an expiration date thereof, which will be described below. The input unit 190 may be realized in the form of a keyboard, a mouse, or a touch screen and may be one of the various well-known input units.

A person in charge of managing an inventory of medicinal products, such as a pharmacist, can register pieces of information, such as an expiration date of a medicinal product, by the barcode reader 150 reading a barcode of the medicinal product or by the input unit 190 performing information-inputting. Thus, quantities of medicinal products and expiration dates of the medicinal products are stored in a medicinal DB 120 through an inventory management module 104.

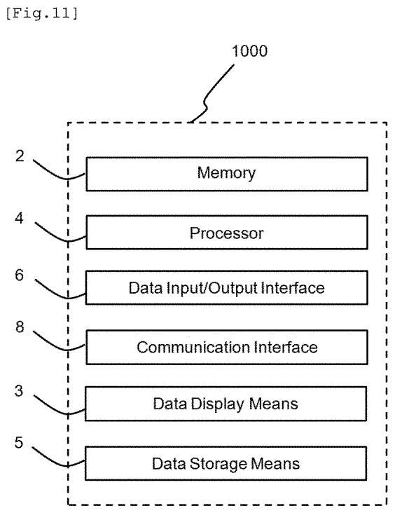

With reference to FIG. 11, from the perspective of hardware, the pharmacy system 1000 according to the present embodiment includes a memory 2 in which one or more commands are stored, and a processor 4 that executes one or more commands stored in the memory 2. The pharmacy system 1000 is a computing apparatus in which a computer program stored on a medium is executed in such a manner as to perform the method of providing notification of an expiration date of a medicinal product. The pharmacy system 1000 according to the present embodiment may include a data input and output interface 6, a communication interface 8, a data display means 3, and a data storage means 5.

From the perspective of functions, the pharmacy system 1000 according to the present embodiment includes an administration module 102 for a general system environment administration, an inventory management module 104 for managing the status of medicinal products in inventory, an external input-and-output module 106 into which audio, characters, and the like are input from the outside or which outputs audio and the like to the outside, and a human-body recognition module 108 that receives signals from first and second human-body detection sensors 142 and 144 that will be described below and recognizes whether or not a person is present in a specific area (a position range) within a pharmacy. The pharmacy system 1000 may further include a payment management module 110 for accepting payment by a customer, an ordering management module 112 for placing an order with a medicinal product supplier and returning a medicinal product to the medicinal product supplier, and a customer management module 114 for managing information on a customer who makes a visit to the pharmacy.

In addition, the pharmacy system 1000 according to the present embodiment includes the medicinal DB 120 for managing medicinal product-related information and may further include a customer DB 130 for managing customer-related information.

The modules in the pharmacy system 1000 described above are configured in terms of functional conception. Of course, some of the modules may be configured to be integrated into one module, or one thereof may be configured to be functionally divided into sub-modules.

The method of providing notification of an expiration date of a medicinal product according to the embodiment of the present invention may be performed independently in the pharmacy system 1000 installed in an individual pharmacy.

As an example, the method of providing notification of an expiration date of a medicinal product is configured to determine whether or not a medicinal product subject to notification is present in the medicinal DB 120 (Step S1) and to output the information about the medicinal product subject to notification in the form of audio guidance through the audio output unit 170 (Step S2).

In Step S1, it is determined whether or not among medicinal products registered as inventory, the medicinal product subject to notification of which an expiration date satisfies a preset notification condition is present.

As an example, the notification condition is set to compare an expiration date of each medicinal product with the current date (time of determination) and to separately manage a medicinal product of which an expiration date is approaching (for example, a medicinal product of which an expiration date or a medication period expires soon).

For example, examples of the separate management may include selection of a medicinal product subject to separate storing, returning or destroying, and assignment of such a medicinal product to another pharmacy that needs the medicinal product, which will be described below.

As an example, in Step S1, among medicinal products registered as inventory, a medicinal product of which an expiration date falls within a preset notification period range is determined as a medicinal product subject to notification that satisfies the notification condition.

The notification period range may be a time range in which the medicinal product subject to notification is separately managed by a pharmacist or the like. All medicinal products are set to have the same notification period range or to have different notification period ranges.

For example, when, for both medicinal product A and medicinal product B, notification period ranges are set in such a manner that notification is provided within six months before an expiration date, it is assumed that an expiration date of medicinal product A is December 15, that an expiration date of medicinal product B is December 10, and that a date on which it is determined whether or not a medicinal product subject to notification is present is June 12. Under this assumption, on June 12, medicinal product B is determined as being the medicinal product subject to notification that satisfies the notification condition.

Multiple notification periods, for example, a period of six months, a period of three months, and a period of one month, may be set for one medicinal product before an expiration date thereof expires.

On the other hand, the notification period range may be a time range that results from considering a medication period for a specific patient.

As an example, in Step S1, at the time of a medicinal product sale to a specific patient, when, among medicinal products registered as inventory, a medicinal product of which an expiration date is determined as exceeding a medication period for the specific patient occurs, the medicinal product is determined as a medicinal product subject to notification that satisfies the notification condition.

Examples of the specific patient include a patient with a chronic illness who has to take medicinal products for a long period of time. The patient with a chronic illness takes a medicinal product for high blood pressure, a medicinal product for heart disease, a medicinal product for diabetes, or the like. In many cases, these medicinal products that the patient will take for a long time period of approximately 30 to 60 days are prepared according to a prescription. When such a specific patient purchases a medicinal product, when the medication period remains, even if it is a part of the medicinal product, the expiration date of the medicinal product should not occur for the protection of the patient.

Therefore, considering a medication period (for example, 60 days) for a specific patient who wants a pharmacist to prepare a medicinal product according to a prescription and/or wants to purchase a medicinal product, a date (for example, May 5) for the medicinal product preparation and/or purchase, and an expiration date (July 1) of the medicinal product, when a medicinal product of which an expiration date is determined as exceeding a medication period for the specific patient occurs, this medicinal product is determined as a medicinal product subject to notification that satisfies the notification condition.

The present embodiment is described with reference to the medication period for the specific patient. However, as another example, considering an expected retaining period (a period during which medicinal products are expectedly used up) for medicinal products for a general patient, a medicinal product of which an expiration date is determined as exceeding the end of the expected retaining period may also be determined as a medicinal product subject to notification that satisfies the notification condition. In this case, the expected retaining period may be set on the basis of questions answered by the general patient and pieces of statistical data.

As an example, Step S1 is performed according to a preset guidance time condition.

The guidance time condition may be a time interval. For example, guidance may be set to be provided at 10 o'clock every day or may be set to be provided immediately after a lunch hour every day. At this time, it is preferable that the guidance time condition is set to be one during pharmacy business hours in such a manner that a pharmacist or the like can receive the audio guidance in Step S2 and thus can check that a medicinal product of which an expiration date is near at hand is held in inventory.

As an example, the pharmacy system 1000 further includes an audio input unit 160. Step S1 is performed through the audio input unit 160 on the basis of audio that is input by a user.

The audio input unit 160 may be realized as a microphone. The external input-and-output module 106 recognizes and analyzes the audio that is input through the microphone, and according to a result of the analysis, performs Step S1.

As an example, the recognition and analysis of the audio that is input through the audio input unit 160 may be made by inputting several audio commands in advance into a known audio analysis engine for setting or by performing audio recognition and analysis learning through the use of artificial intelligence (AI). To this end, the pharmacy system 1000 may further include an artificial intelligence module (not illustrated).

For example, when “Tell me a medicinal product subject to notification as of today”, a pharmacist talks into the audio input unit 160, the pharmacy system 1000 performs Step S1 with a current date as a reference date. When “Tell me a medicinal product subject to notification as of (a particular date)”, the pharmacist talks into the audio input unit 160, the pharmacy system 1000 performs Step S1 with the particular date as the reference date.

In Step S2, the audio guidance in the form of an audio message provides at least one of the following pieces of information on the medicinal product subject to notification: a name, an inventory quantity, and an expiration date.

For example, when, in Step S1, 50 of 200 medicinal products “A” are determined as medicinal products subject to notification, an audio message saying “expiration dates of 50 medicinal products “A” are 60 days away” may be output as the audio guidance.

For example, when, in Step S1, 100 medicinal products “B” are all determined as medicinal products subject to notification, an audio message saying “expiration dates of 100 medicinal products “B” are December 10” may be output as the audio guidance.

It is preferable that a pharmacist can know all of the medicinal product name, inventory quantity, and expiration date in order to separate or intensively manage the medicinal products subject to notification. However, if there are many medicinal products subject to notification, it may be difficult to hear all the information by voice. Therefore, it is desirable to provide the minimum information that the medicinal products subject to notification exists, and to additionally check it by other output methods such as screen display or printing, which will be described later.

As an example, the pharmacy system 1000 may further include a screen output unit 180. The method of providing notification of an expiration date of a medicinal product, as illustrated in FIG. 3, may be configured to further include Step S21 of screen-displaying the information about the medicinal product subject to notification through the screen output unit 180.

The screen output unit 180 may be a screen display device that is provided in the POS terminal 140 and is capable of displaying a payment process, and may be a screen display device that is provided separately from the POS terminal 140 and displays medicinal product information at the request of a pharmacist or the like (or automatically). The screen output unit 180 may be an image display device, such as a monitor or a TV. The information on the medicinal products subject to notification displayed on the screen may be controlled to be displayed on a tablet or smartphone that is connected to the pharmacy system 1000 and possessed by a pharmacist.

The screen display is provided as a supplement to the audio guidance. With the audio guidance, a pharmacist can know whether or not a medicinal product of which an expiration date is near at hand is present. Furthermore, the pharmacist can obtain detailed information through the screen output unit 180 and thus can manage medicinal product inventory in an easier manner.

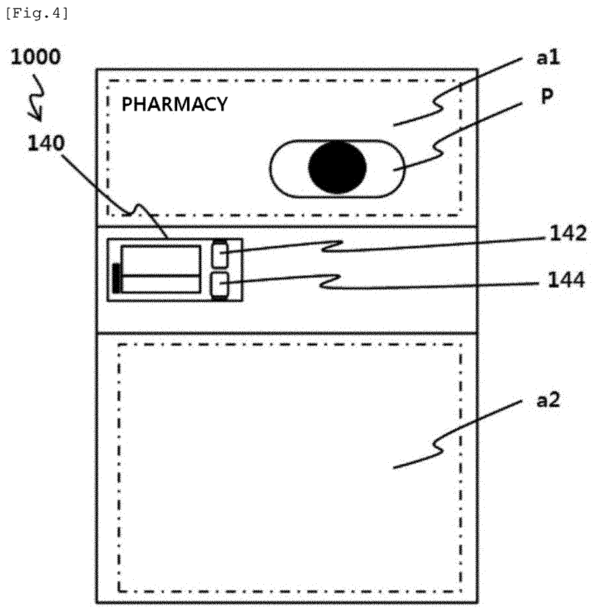

FIG. 4 is a schematic diagram illustrating the inside of a pharmacy in which the pharmacy system according to the embodiment of the present invention is installed.

As an example, the POS terminal 140 includes a first human-body detection sensor 142 configured to detect a pharmacist-side area a1 and a second human-body detection sensor 144 configured to detect a customer-side area a2.

The first and second human-body detection sensors 142 and 144 may be configured as a (short-distance) infrared sensor, an ultrasonic sensor, a microwave sensor, a capacitive sensor, or the like. Various types of known sensors that are capable of detecting whether or not a human body is present are available as the first and second human-body detection sensors 142 and 144.

The pharmacist-side area a1 indicates an area (for example, the vicinity of a POS terminal) where an employee in charge of inventory management in a pharmacy, such as a pharmacist, normally works. The customer-side area a2 indicates an area where a customer who makes a visit to the pharmacy stays.

The pharmacy system 1000 performs Step S2 when the first human-body detection sensor 142 detects a human body P and the second human-body detection sensor 144 does not detect a human body P.

The detection of a human body P through the first human-body detection sensor 142 indicates that the human body P is present in the pharmacist-side area a1. This means that the employee in charge of inventory management in the pharmacy, such as a pharmacist, is present in the corresponding area. In addition, the non-detection of the human body P through the second human-body detection sensor 144 indicates that a customer or the like is not present in the customer-side area a2.

In this situation, for example, a pharmacist is present in the vicinity of the POS terminal 140 and a customer is not present. In this case, while provided with guidance on a medicinal product subject to notification, the pharmacist is not disturbed by the customer. Thus, the pharmacist can completely get information about an expiration (for example, information on a medicinal product subject to notification) without any missing part thereof.

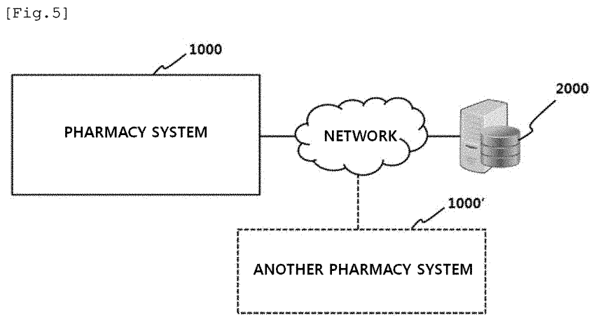

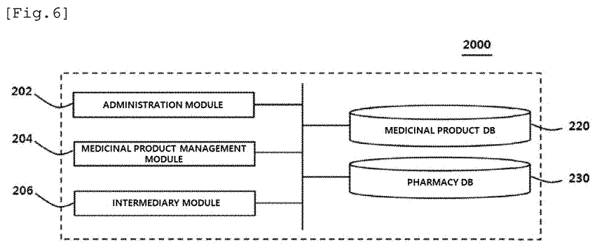

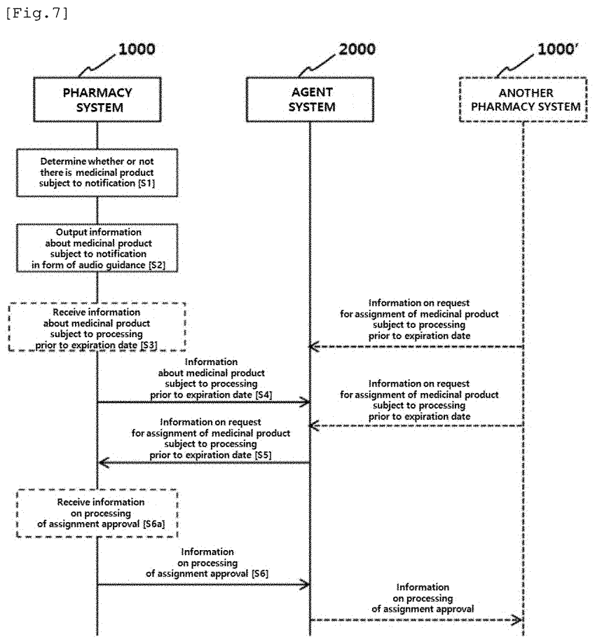

FIG. 5 is a diagram illustrating a configuration of a system in which a method of providing notification of an expiration date of a medicinal product according to another embodiment of the present invention is performed. FIG. 6 is a diagram illustrating a configuration of an agent system according to an embodiment of the present invention. FIG. 7 is a flowchart illustrating a method of providing notification of an expiration date of a medicinal product according to still another embodiment of the present invention.

As an example, as illustrated in FIG. 5, the pharmacy system 1000 may operate in conjunction with an agent system 2000 via a network.

A network according to the present embodiment refers to a normal communication network, such as the Internet or an intranet. Examples of this network include wired and wireless networks.

The agent system 2000, as illustrated in FIG. 6, includes an administration module 202 for a general system environment administration, a medicinal product management module 204 for managing information about a medicinal product subject to processing prior to an expiration date thereof, which is transmitted from the pharmacy system 1000, and a intermediary module 206 that transmits the information about the medicinal product subject to processing prior to the expiration date thereof to another pharmacy system 1000′ and receives a request from the another pharmacy system 1000′ for assignment of the medicinal product subject to processing prior to the expiration date thereof.

In addition, the agent system 2000 according to the present invention includes a medicinal product DB 220 for managing medicinal product-related information and a pharmacy DB 230 for managing pharmacy-related information.

As illustrated in FIG. 7, the method of providing notification of an expiration date of a medicinal product according to the present embodiment is configured to include Step S3 of receiving input about the medicinal product subject to processing prior to the expiration date thereof, among the medicinal products subject to notification about which the information is output in the form of audio guidance, Step S4 of transmitting the information about the input medicinal product subject to processing prior to the expiration date thereof to the agent system 2000, Step S5 of receiving from the agent system 2000 information about the pharmacy system 1000′ that makes the request for the assignment of the medicinal product subject to processing prior to the expiration date thereof, and Step S6 of transmitting an approval of the assignment of the medicinal product subject to processing prior to the expiration date thereof, the request for whose assignment is made, to the agent system 2000 when an approval of the assignment of the medicinal product subject to processing prior to the expiration date thereof is input for processing.

In Step S3, among the medicinal products subject to notification, about which the pieces of information are output in the form of audio guidance through Steps S1 and S2, the medicinal product subject to processing prior to the expiration date thereof is input through the input unit 190.

The medicinal product subject to processing prior to the expiration date is selected based on the expiration date for the purpose of assigning (transferring) the medicinal product before the expiration date expires. The pharmacist may select and input a medicinal product to be sold to another pharmacy instead of returning or discarding it to a medicinal product supplier, etc., among the medicinal products subject to notification.

In Step S4, the information about the medicinal product subject to processing prior to the expiration date thereof is transmitted to the agent system 2000 that operates in conjunction with the pharmacy system 1000 via a network.

In this case, in Step S4, the information about the medicinal product subject to processing prior to the expiration date thereof includes at least one of a price condition for assigning the medicinal product and an expiration date condition for assigning the medicinal product.

The price condition for assigning the medicinal product may be set as a condition that a discount rate is applied, considering the remaining effectiveness period for the medicinal product. The expiration date condition for assigning the medicinal product may be set as a condition that assigning is possible only if the assigning is possible within a certain period in consideration of the effectiveness period for the medicinal product.

The agent system 2000 transmits the information about the medicinal product subject to processing prior to the expiration date thereof to another pharmacy system 1000′ and receives the request for the assignment of the medicinal product subject to processing prior to the expiration date thereof from the another pharmacy system 1000′.

In this case, in a state when the agent system 2000 does not transmit the information about the medicinal product subject to processing prior to the expiration date thereof to another pharmacy system 1000′, information on the request for the assignment of the medicinal product may be received in advance from the another pharmacy system 1000′. For example, it is also possible that another pharmacy system 1000′ in another pharmacy that has a shortage of the medicinal products in inventory transmits in advance request information that it will receive the medicinal product that is selected as the medicinal product subject to processing prior to the expiration date thereof in any other pharmacy system, to the agent system 2000.

However, when considering an assignment price of or an assignment expiration date of the medicinal product subject to processing prior to the expiration date thereof, it may be disadvantageous for the another pharmacy to unconditionally receive the medicinal product subject to processing prior to the expiration date thereof. Thus, it is preferable, after the another pharmacy system 1000′ receives the information about the medicinal product subject to processing prior to the expiration date thereof from the agent system 2000, that a pharmacist who works for the another pharmacy decides whether or not the assignment request is made, considering the price condition for assignment and/or the expiration date condition for assignment.

In Step S5, the agent system 2000 transmits information about the another pharmacy system 1000′ that makes the request for the assignment of the medicinal product subject to processing prior to the expiration date thereof, to the pharmacy system 1000.

In the pharmacy system 1000, the approval of the assignment of the medicinal product subject to processing prior to the expiration date thereof, for whose assignment the request is made, is input by the pharmacist through the input unit 190 for processing (Step S6a).

Next, information on the approval of the assignment of the medicinal product subject to processing prior to the expiration date thereof, for whose assignment the request is made, is transmitted to the agent system 2000 (Step S6). The transmission of the information on the approval of the assignment to the another pharmacy system 1000′ by the agent system 2000 completes an assignment contract between the pharmacy that transmits the medicinal product subject to processing prior to the expiration date thereof and the pharmacy that makes the request for the assignment. According to the contract, additional processes, such as transfer of the medicinal product subject to processing prior to the expiration date thereof, may be performed.

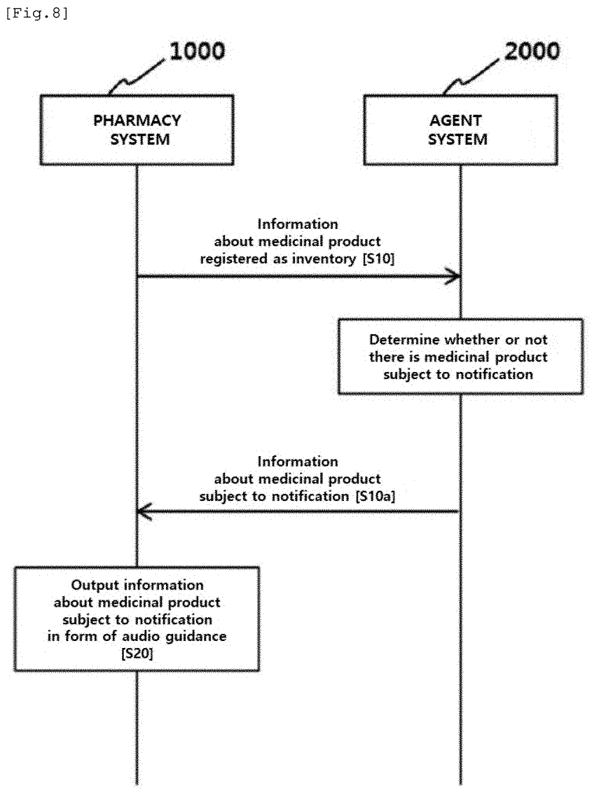

FIG. 8 is a flowchart illustrating a method of providing notification of an expiration date of a medicinal product according to still another embodiment of the present invention.

The pharmacy system 1000 that includes the POS terminal 140 and the audio output unit 170 performs the method of providing notification of an expiration date of a medicinal product according to the embodiment of the present invention, in conjunction with the agent system 2000 via a network.

In the method of providing notification of an expiration date of a medicinal product, the pharmacy system 1000 is configured to transmit all pieces of information about the registered medicinal products to the agent system 2000, the agent system 2000 is configured to make a determination as to the expiration date, and the pharmacy system 1000 is configured to provide the audio guidance according to a result of the determination.

To this end, in Step S10, the information about the medicinal product registered as inventory in the pharmacy system 1000 is transmitted to the agent system 2000.

The agent system 2000 manages information on an inventory in each pharmacy system on the basis of the transmitted information.

The agent system 2000 determines whether or not, among the medicinal products registered as inventory, a medicinal product subject to notification, of which an expiration date satisfies a preset notification condition, is present.

When it is determined that among the medicinal products registered as inventory, the medicinal product subject to notification, of which the expiration date satisfies the preset notification condition, is present, the information about the medicinal product subject to notification is transmitted from the agent system 2000 to the pharmacy system 1000 (Step S10a).

Next, for the audio guidance, the pharmacy system 1000 outputs the information about the medicinal product subject to notification in the form of an audio message through the audio output unit 170 (Step S20).

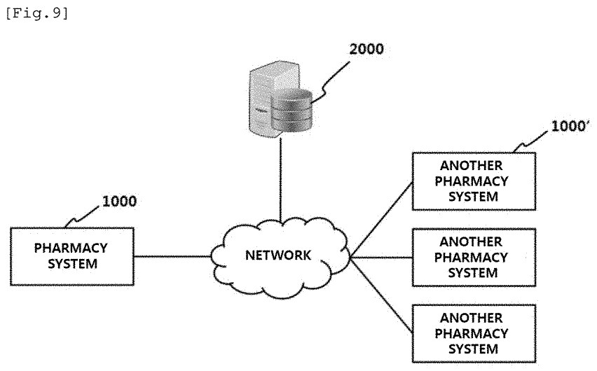

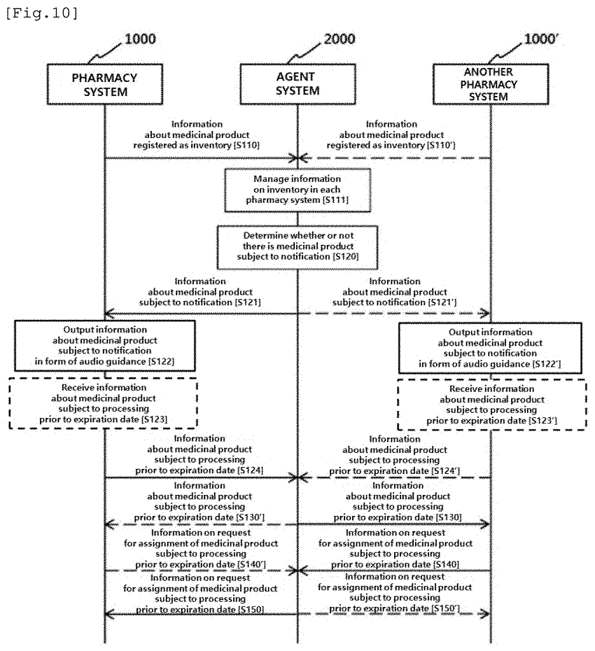

FIG. 9 is a diagram illustrating a configuration of a system in which a method of providing notification of an expiration date of a medicinal product according to still another embodiment of the present invention is performed. FIG. 10 is a flowchart illustrating a method of providing notification of an expiration date of a medicinal product according to still another embodiment of the present invention.

The method of providing notification of an expiration date of a medicinal product according to the embodiment of the present invention is performed in the agent system 2000 that operates in conjunction with the pharmacy system 1000 which includes the POS terminal 140 and the audio output unit 170, via a network.

In the method of providing notification of an expiration date of a medicinal product according to the present embodiment, as illustrated in FIG. 10, the agent system 2000 receives the information about the medicinal product registered as inventory, from the pharmacy system 1000, in Step S110, and manages the information on the inventory in each pharmacy system on the basis of the received information, in Step S111.

It is determined that among the medicinal products registered as inventory in each pharmacy system, the medicinal product subject to notification, of which the expiration date satisfies the preset notification condition, is present (Step S120). The information about the medicinal product subject to notification is transmitted to the corresponding pharmacy system 1000 (Step S121).

In the pharmacy system 1000, the information about the medicinal product subject to notification is output in the form of audio guidance through the audio output unit 170 (Step S122).

In the pharmacy system 1000, the information about the medicinal product subject to processing prior to the expiration date thereof, which is input by a pharmacist using the input unit 190, is received (Step S123).

The medicinal product subject to processing prior to the expiration date thereof is a medicinal product that, among the medicinal products subject to notification that satisfy the preset notification condition, which are among the medicinal products registered as inventory in each pharmacy system, is input as the medicinal product subject to processing prior to the expiration date thereof. The medicinal product subject to processing prior to the expiration date thereof is input on the basis of the information about the medicinal product subject to notification, which is output in the form of audio guidance through the audio output unit 170 in the pharmacy system 1000.

The medicinal product subject to processing prior to the expiration date thereof is selectively input in such a manner that processing (for example, returning or assigning) is performed before the expiration date thereof expires.

The agent system 2000 receives the information about the medicinal product subject to processing prior to the expiration date thereof from the pharmacy system 1000 (Step S124) and provides the information about the medicinal product subject to processing prior to the expiration date thereof to another pharmacy system 1000′ (Step S130).

As an example, in step S130, the information about the medicinal product subject to processing prior to the expiration date thereof is configured to be provided only to another pharmacy system 1000′, which has previously registered its intention to receive the corresponding medicinal product.

When the pharmacy system 1000 provides the information about the medicinal product to all other pharmacy systems 1000′, as is the case when a spam e-mail message is received, a pharmacist and a manager of another pharmacy system 1000′, that has no intention to receive the medicinal product, are burdened with excessive information about the medicinal product. Therefore, it is preferable that the information about the medicinal product subject to processing prior to the expiration date thereof is provided only to another pharmacy system 1000′ that in advance registers its intention to receive the corresponding medicinal product.

As another example, in Step S130, the information about the medicinal product subject to processing prior to the expiration date is provided only to another pharmacy system 1000′ that, among the pharmacy systems 1000′ that register their intentions to receive the corresponding medicinal product, is located at the same area as the pharmacy system 1000.

In order to complete the processes of assigning and receiving the medicinal product subject to processing prior to the expiration date thereof, a process of transmitting the medicinal product subject to processing prior to the expiration date thereof from a pharmacy in which the pharmacy system 1000 is installed to another pharmacy in which another pharmacy system 1000′ is installed is required. When a pharmacy that is an assignee is located remotely from a pharmacy that is an assignor, various expenses, such as handling charge and transportation expenses, may occur. Therefore, it is preferable that an area to which the intention to receive the corresponding medicinal product is transmitted is limited to a condition that the another pharmacy system 1000′ in the another pharmacy that is an assignee is located in the same area as the pharmacy system 1000 in the pharmacy that is an assignor.

As an example, the method of providing notification of an expiration date of a medicinal product is configured to further include Step S140 in which the agent system 2000 receives the information about the request of the assignment of the medicinal product subject to processing prior to the expiration date thereof from another pharmacy system 1000′, and Step S150 in which the agent system 2000 provides the information on the request from the another pharmacy system 1000′ for the assignment of the medicinal product to the pharmacy system 1000 that transmits the information about the medicinal product subject to processing prior to the expiration date thereof.

Details of assignment approval processing and the like, which will be performed after, are as described above according to the embodiment, and therefore, descriptions thereof are omitted.

In FIG. 10, for facilitating an understanding of the invention, a path along which the information about the medicinal product registered as inventory in the pharmacy system 1000 is transmitted is indicated by a solid line, and a path along which the information about the medicinal product registered as inventory in another pharmacy system 1000′ is transmitted is indicated by a dotted line. Steps of transmitting the information about the medicinal product registered as inventory in another pharmacy system 1000′ is indicated by adding apostrophes (′) to the ends of character references, respectively, of steps of transmitting the information about the pharmacy system 1000.

A method of providing notification of an expiration date of a medicinal product according to an embodiment of the present invention is provided in the form of a computer program stored in a computer-readable recording medium such that the method of providing notification of an expiration date of a medicinal product can be performed in conjunction with a piece of hardware. It is also possible that for distribution, the method of providing notification of an expiration date of a medicinal product is recorded on a computer-readable medium so that the method can be executed by a computer.

To this end, embodiments of the present invention include program instructions for performing various computer-implemented operations and computer-readable media including the same. The computer-readable medium may include program instructions, data files, data structures, or the like alone or in combination. The media may be specially designed and constructed for the present invention, or may be known and usable to those skilled in computer software. Examples of computer-readable recording media includes magnetic media, such as hard disks, floppy disks, and magnetic tapes, optical recording media such as CD-ROM, DVD, USB drives, magnetic-optical media such as floptical discs, and hardware device specially configured to store and execute program instructions such as ROM, RAM, flash memory, etc. Examples of program commands includes machine code like one produced by the compiler, as well as high-level language code that can be executed by a computer using an interpreter, etc.

Preferable embodiments of the present invention are described in a focused manner with reference to the accompanying drawings. From the specification, it is apparent to a person of ordinary skill in the art that various modifications to the present invention are possible in an obvious manner without departing from the scope of the present invention. Therefore, these many modification examples fall within the scope of the present invention that is defined by the following claims.

1. A method of providing notification of an expiration date of a medicinal product, which is performed in a pharmacy system that includes a POS terminal and an audio output unit, the method comprising:
1) determining whether or not, among medicinal products registered as inventory, there is a medicinal product subject to notification, of which an expiration date satisfies a preset notification condition; and
2) outputting information about the medicinal product subject to notification in the form of audio guidance through the audio output unit.

2. The method according to claim 1, wherein the step 1) is automatically performed according to a preset guidance time condition.

3. The method according to claim 1, wherein the pharmacy system further includes an audio input unit, and
the step 1) is performed on the basis of audio that is input by a user through the audio input unit.

4. The method according to claim 1, wherein in the step 1), among the medicinal products registered as inventory, a medicinal product of which an expiration date falls within a preset notification period range is determined as the medicinal product subject to notification that satisfies the notification condition.

5. The method according to claim 1, wherein in the step 1), at the time of a medicinal product sale to a specific patient, when, among the medicinal products registered as inventory, a medicinal product of which an expiration date is determined as exceeding a medication period for the specific patient occurs, the medicinal product is determined as the medicinal product subject to notification that satisfies the notification condition.

6. The method according to claim 1, wherein the pharmacy system further includes a screen output unit, the method further comprising:
21) screen-displaying the information about the medicinal product subject to notification through the screen output unit.

7. The method according to claim 1, wherein the POS terminal comprises a first human-body detection sensor configured to detect a pharmacist-side area and a second human-body detection sensor configured to detect a customer-side area, and
wherein the pharmacy system performs the step 2) when the first human-body detection sensor detects a human body and the second human-body detection sensor does not detect the human body.

8. The method according to claim 1, wherein the pharmacy system operates in conjunction with an agent system via a network, the method further comprising:
3) receiving input about a medicinal product subject to processing prior to an expiration date, among the medicinal products subject to notification about which the information is output in the form of audio guidance;
4) transmitting the information about the input medicinal product subject to processing prior to the expiration date to the agent system;
5) receiving information about another pharmacy system that makes a request for assignment of the medicinal product subject to processing prior to the expiration date, from the agent system; and
6) transmitting an approval of the assignment of the medicinal product subject to processing prior to the expiration date to the agent system, when the approval of the assignment of the medicinal product subject to processing prior to the expiration date is input for processing, the medicinal product subject to processing prior to the expiration date being requested for assignment.

9. The method according to claim 8, wherein in the step 4), at least one of a price condition for assigning the medicinal product and an expiration date condition for assigning the medicinal product is included in the information about the medicinal product subject to processing prior to the expiration date.

10. A method of providing notification of an expiration date of a medicinal product, which is performed in a pharmacy system that includes a POS terminal and an audio output unit and operates in conjunction with an agent system via a network, the method comprising:
1) transmitting information about a medicinal product registered as inventory in the pharmacy system to the agent system,
the agent system managing information on an inventory in each pharmacy system on the basis of the received information; and
2) when it is determined that, among medicinal products registered as inventory, there is a medicine product subject to notification, of which the expiration date satisfies a preset notification condition,
outputting information about the medicinal product subject to notification in the form of audio guidance through the audio output unit,
the determination being made in the agent system and the information about the medicinal product subject to notification being transmitted from the agent system to the pharmacy system.

11. A method of providing notification of an expiration date of a medicinal product, which is performed in an agent system that operates via a network in conjunction with a pharmacy system that includes a POS terminal and an audio output unit, the method comprising:
1) receiving information about a medicinal product registered as inventory from the pharmacy system and managing information on the inventory in each pharmacy system on the basis of the received information;
2) receiving information about a medicinal product subject to processing prior to an expiration date from the pharmacy system,
the medicinal product subject to processing prior to the expiration date being a medicinal product input as a medicinal product subject to processing prior to the expiration date among medicinal products subject to notification, of which an expiration date satisfies a preset notification condition, and being medicinal products registered as inventory in each pharmacy system,
and the medicinal product subject to processing prior to the expiration date being input on the basis of information about the medicinal product subject to notification, which is output in the form of audio guidance through the audio output unit in the pharmacy system; and
3) providing the information about the medicinal product subject to processing prior to the expiration date to another pharmacy system.

12. The method according to claim 11, further comprising:
4) receiving information on a request for assignment of the medicinal product subject to processing prior to the expiration date from the another pharmacy system; and
5) providing the information on the request for the assignment from the another pharmacy system to the pharmacy system that transmits the information about the medicinal product subject to processing prior to the expiration date.

13. The method according to claim 11, wherein in the step 3), the information about the medicinal product subject to processing prior to the expiration date is provided only to another pharmacy system that in advance registers its intention to receive the medicinal product.

14. The method according to claim 13, wherein in the step 3), the information about the medicinal product subject to processing prior to the expiration date is provided only to another pharmacy system that, among other pharmacy systems that in advance register their intentions to receive the medicinal product, is located in the same area as the pharmacy system.

15. A computer-readable recording medium on which a computer program for performing a method of providing notification of an expiration date of a medicinal product in a state of being combined with a pharmacy system that includes a POS terminal and an audio output unit is recorded, the method comprising:
determining whether or not, among medicinal products registered as inventory, there is a medicinal product subject to notification, of which an expiration date satisfies a preset notification condition; and
outputting information about the medicinal product subject to notification in the form of audio guidance through the audio output unit.
